(19) United States
(12) Patent Application Publication
(10) Pub. No.: US 2023/0293482 A1
(43) Pub. Date: Sep. 21, 2023

(54) CENTRAL NERVOUS SYSTEM POTENTIATING COMPOSITION

(71) Applicant: SHIBAURA INSTITUTE OF TECHNOLOGY, Tokyo (JP)

(72) Inventors: Naomi OSAKABE, Tokyo (JP); Yasuyuki FUJII, Tokyo (JP); Taiki FUSHIMI, Tokyo (JP)

(73) Assignee: SHIBAURA INSTITUTE OF TECHNOLOGY, Tokyo (JP)

(21) Appl. No.: 17/913,259

(22) PCT Filed: Mar. 24, 2021
(86) PCT No.: PCT/JP2021/012174

§ 371 Date: Apr. 18, 2023

(30) Foreign Application Priority Data
Mar. 24, 2020 (JP) 2020-053459

(51) Int. Cl. A61K 31/353; A61P 25/28; A61K 9/00

(52) U.S. Cl. CPC A61K 31/353; A61P 25/28; A61K 9/0053

(57) ABSTRACT

It is an object of the present invention to provide a safe and effective central nerve-activating composition, The present invention provides a central nerve-activating composition comprising, as an active ingredient, 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dthydroxyphenyl)-3,4-dihydro-2H-chromene-3,5,7-triol are condensed by a 4β→8 bond, or a salt, hydrate, solvate or polymorph thereof, wherein the central nerve-activating composition adjusts autonomic nerve activity via production of superoxide and/or hydrogen peroxide.

### TECHNICAL FIELD

The present invention relates to a central nerve-activating composition, and an agent for producing superoxide and/or hydrogen peroxide, both of which comprise 2-(3,4-dihydroxyphenyl)chromenylium-3,5,7-triol 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or poly morph thereof.

### BACKGROUND ART

At present, 7000 types or more of polyphenols have been identified, and these polyphenols are present as glycosides, or some of them are present as aglycones, in plants. Such polyphenol is a plant pigment component exhibiting dark brown, red or purple color, and has astringency. Plants synthesize polyphenols via shikimic acid pathways, and eliminate active oxygen generated upon ultraviolet irradiation or energy production as follows (Non-Patent Document 1).

[Formula 1]

RH→R· (1)

R·+O2→R—OO· (2)

R—OO·+RH→R—OOH+R· (3)

R—OO·+RH→R—OOH+P· (4)

P·→Oxidative decomposition

R: Plant component P: Polyphenol

The above reactions (1) to (3) are oxidation reactions occurring together with electron transfer upon ultraviolet irradiation or upon energy production, and the above reaction (4) is an antioxidant reaction. Hence, it has been considered that polyphenols mainly have antioxidant action, and thus, the development of the polyphenols as antioxidants has been promoted (Patent Documents 1 to 4).

In plant bodies, polyphenols are stably stored in vacuoles exhibiting weak acidity. However, when vacuoles are damaged, the poly phenols are discharged to the cytoplasm, and are oxidatively decomposed due to pH change (Non-Patent Document 1), or by polyphenol oxidase, so that they are browned Ton-Patent Document 2).

The polyphenol is a compound comprised also in food products, and humans ingest the polyphenols as food products. A large number of epidemiological researches have demonstrated that, when patients with cardiovascular diseases such as myocardial infarction, chronic heart disease, cerebral infarction or stroke, or with all other diseases, frequently ingest food products of plant origin that are known to abundantly comprise polyphenols, the risk of death is reduced (Non Patent Document 3).

It has been known that the bioabsorbable property of polyphenols is extremely low in living human bodies. Among such polyphenols, flavonoids or simple phenols that are aglycones having a relatively low molecular weight (molecular weight<400) are absorbed by, at maximum, about 10%, through the small intestine. However, a majority thereof is metabolized in the process in which the aglycones pass through the small intestine and/or the liver, and they are present in the form of metabolites in the peripheral blood. In addition, flavonoid polymers are hardly absorbed through the alimentary canal, and the concentration of the flavonoid polymers in the blood or tissues is extremely low (Non-Patent Document 4).

Meanwhile, to date, it has been often reported that vascular endothelial function is improved 2 to 4 hours after ingestion of food products with high polyphenol content. In order to clarify this phenomenon observed in humans, the present inventors have administered a plant extract abundantly containing polyphenols to laboratory animals, and have then observed the influence of the polyphenols on the peripheral blood flow thereof. As a result, the present inventors have confirmed that the blood flow increased in the cremasteric arteriole immediately after the administration. In addition, the present inventors have administered to the laboratory animals, such a plant extract abundantly containing polyphenols in combination with carvedilol that is a non-specific adrenoreceptor inhibitor. As a result, the aforementioned change was significantly suppressed (Non-Patent Document 5). From these results, it has been revealed that polyphenols promote sympathetic nerve activity and increase the peripheral blood flow.

When humans ingest polyphenols, the ingested polyphenols are not absorbed but are excreted, and the polyphenols pass through the alimentary canal (Non-Patent Document 6). Thus, it has been conceived that the target molecules of the polyphenols are present in the alimentary canal. When humans ingest polyphenols, they feel strong astringency. It has been conceived that the stimulation is recognized by the sensory nerve.

In view of the foregoing, the present inventors have administered a plant extract abundantly containing polyphenols to sensory- nerve-removed rat models, and have then measured the peripheral blood flow. As a result, no change was observed, From these results, it was revealed that the sensory nerve is involved in the expression of the action of polyphenols to promote the sympathetic nerve activity (Non-Patent Document 7).

It has been known that capsaicin or allyl isothiocyanate that is a pungent component ignites the sensory nerve distributing in the alimentary canal including the oral cavity, and that the stimulation is transmitted to the central nerve, so that the sympathetic nerve activity is promoted. Capsaicin serving as a pungent component is a ligand of Transient Receptor Potential Vanilloid 1 (TRPV1) expressed on the sensory nerve, and the stimulation thereof is transmitted to the central nerve via the secondary nerve. In response to this stimulation, a stress response reaction occurs in the hypothalamus, and at the same time, the sympathetic nerve activity is promoted via the brainstem, Consequently, physiological fluctuations such as sweating, increased heart rate, increased blood pressure and increased blood flow are induced to peripheral areas. Moreover, allyl isothiocyanate contained in -wasabi mustard is a ligand of Transient Receptor Potential Ankylin 1 (TRPA1), and menthol contained in mint is a ligand of Transient Receptor Potential cation channel subfamily Melastatin 8 (TRPM8). These substances also promote the sympathetic nerve activity by the same mechanism as that described above (Non-Patent Document 8).

TRP channel is a calcium ion channel having 6 transmembrane domains, and is classified into 7 subfamilies, based on the similarity of amino acid sequence or molecular structure. That is, 7 subfamilies, namely, TRPV, TRPC, TRPM, TRPP, TRPML, TRPN, and TRPA are present. In humans, 6 out of the 7 subfamilies and 27 channels are present, and such TRP channels are distributed as sensors receiving many chemical or physical stimulations in various organs including alimentary canal, skin, and mucous membrane, and are deeply associated with the biological function (Non-Patent Document 9).

When strong astringency is felt upon ingestion of polyphenols, or when a plant extract abundantly containing polyphenols is ingested, promotion of the sympathetic nerve activity, such as blood flow-increasing action or energy production-promoting action, is observed, as with other TRP ligands. Hence, it has been conceived that some or a majority of 700 types of polyphenols would be likely to be ligands of the TRP channels.

On the other hand, one of the serious problems of modern society may be maladaptation to society, such as frailty or social withdrawal, and such social maladaptation causes serious socioeconomic loss. The social maladaptation is a state in which a mental aspect such as a depressive state or a decline in energy and/or cognitive function, a physical aspect such as a decline in muscle mass or mobility impairment, and a social aspect such as social isolation are comprehensively weakened. Insufficient central nerve function induces disorders such as sleep rhythm disorder, orthostatic dysregulation, depression, or cognitive dysfunction. In addition, such insufficient central nerve function further induces autonomic nervous system disorder, which enhances the risk of complication of diseases such as obesity, hypertension, hyperlipidemia, diabetes, sarcopenia, and disuse muscular atrophy.

### PRIOR ART DOCUMENTS

### Patent Documents

Patent Document 1: JP Patent Publication (Kokai) No, 2000-060485 A
Patent Document 2: CA2663970C
Patent Document 3: U.S. Pat. No. 3,784,480A
Patent Document 4: U.S. Pat. No. 8,168,211B2

### Non-Patent Documents

Non-Patent Document 1: Yang J. Cohen Stuart M A, Kamperman M Jack of all trades: versatile catechol crosslinking mechanisms. Chem Sac Rev, 43,8271,014
Non-Patent Document 2: Sullivan, M. L. Beyond brown: polyphenol oxidases as enzymes of plant specialized metabolism. Frontiers in plant science 5, 783, 2014.
Non-Patent Document 3: Cory, H., Passarelli, S., Szeto, J., Tamez, M. &. Mattei, J. The Role of Polyphenols in Human Health and Food Systems: A Mini-Review. 5, 87, 2018.
Non-Patent Document 4: Gonzales, G. B. et al. Flavonoid interactions during digestion, absorption, distribution and metabolism: a sequential structure-activity/property relationship-based approach in the study of bioavailability and bioactivity. Drug metabolism reviews 47, 2015.
Non-Patent Document 5: Saito, A, et al. Onset of a hypotensive effect following ingestion of flavan 3-ols involved in the activation of adrenergic receptors. Free radical biology & medicine 99, 584-592, 2016.
Non-Patent Document 6: Osakabe, N. & Terao, J. Possible mechanisms of postprandial physiological alterations following flavan 3-ol ingestion. Nutr Rev 76, 174-186, 2018.
Non-Patent Document 7: N. Watanabe, A. Yasuoka, K. Inagawa, N. Kamio, R. Ebe, T. Suzuki, K. Abe, N. Osakabe; Recognition Mechanism 1 of Hardly Absorbable Polyphenols in Alimentary Canal—Gene Expression Analysis of Dorsal Root Ganglions of Sensory Nerve-Removed Rat Models—The 2015 Annual Conference of the Japan Society for Bioscience, Biotechnology and Agrochemistry
Non-Patent Document 8: Moran, M. M. TRP Channels as Potential Drug, Targets. Annual review of pharmacology and toxicology 58, 309-330, 2018.
Non-Patent Document 9: Geffeney S. Sensory Mechanotransduction and Thermotransduction in Invertebrates in Neurobiology of TRP Channels. 2nd edition. Emir TLR, Edt. CRC Press/Taylor & Francis, 2017.

### SUMMARY OF INVENTION

### Object to be Solved by the Invention

Conventionally, in order to prevent or improve such maladaptive conditions to the society, comprehensive approaches such as approach from mental health, cognitive behavioral therapy, exercise therapy, nutritional therapy and drug therapy have been conducted. However, a sufficient improvement method has not yet been established.

It is an object of the present invention to provide a safe and effective central nerve-activating composition for preventing or treating maladaptive conditions to society and diseases whose onset are induced by such maladaptive conditions. The maladaptive conditions to the society may include hypersomnia, overreaction to stress, depressive state, decreased memory/learning ability, decreased energy metabolism, damage to the circulatory system, and atrophy of the skeletal muscle. Examples of the disease induced by such maladaptive conditions may include sleep rhythm disorder, orthostatic dysregulation, mild cognitive impairment, dementia, chronic wounds, obesity, hypertension, hyperlipidemia, diabetes, sarcopenia, and disuse muscular atrophy.

### Means for Solving the Object

As a result of intensive studies conducted directed towards achieving the aforementioned object, the present inventors have found that 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, is able to promote autonomic nerve activity via production of superoxide and/or hydrogen peroxide, and is able to activate the central nerve, thereby completing the present invention.

Specifically, the present invention relates to the following features.

<1> A central nerve-activating composition comprising, as an active ingredient, 2-(3,4-dihydroxyphenyl)chromenylium-3,5,7-triol 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, wherein the central nerve-activating composition adjusts autonomic nerve activity via production of superoxide and/or hydrogen peroxide.
<2> The central nerve-activating composition according to the above <1>, which is used for the purpose of achieving at least one of wakening, improvement of concentration, improvement of working memory, improvement of cognitive function, improvement of stress resilience, beiging of adipose tissues, muscular hypertrophy, and neovascularization, or for the purpose of preventing or treating at least one of sleep rhythm disorder, orthostatic dysregulation, depressive symptoms, mild cognitive impairment, dementia, disuse muscular atrophy, sarcopenia, obesity, hypertension, hyperlipidemia, and diabetes.
<3> The central nerve-activating composition according to the above <1> or <2>, which is administered by oral ingestion and/or application and/or aspiration.
<4> The central nerve-activating composition according to any one of the above <1> to <3>, wherein the active ingredient thereof is administered to a human at a daily dose of 0.1 μg to 1000 mg.
<5> The central nerve-activating composition according to any one of the above <1> to <4>, which produces hydrogen peroxide in one or more of the oral cavity, esophagus, small and large intestine, skin, and mucous membrane, then activates the sensory nerve as a result that the produced hydrogen peroxide is recognized by the superoxide/hydrogen peroxide domain of a transient receptor potential (TRP) expressed on the sensory nerve, and then promotes the central nerve activity via the secondary nerve, so that the central nerve-activating composition promotes the autonomic nerve activity.
<6> The central nerve-activating composition according to the above <5>, wherein the transient receptor potential (TRP) is Transient Receptor Potential Ankyrin 1 (TRPA1), Transient Receptor Potential Melastatin 1 (TRPM1), Transient Receptor Potential melastatin 2 (TRPM2), Transient Receptor Potential Cation channel 1 (TRPC1), Transient Receptor Potential Cation channel 3 (TRPC3), Transient Receptor Potential Cation channel 4 (TRPC4), Transient Receptor Potential Cation channel 5 (TRPC5), Transient Receptor Potential Vaniloid 1 (TRPV1), Transient Receptor Potential Vaniloid 3 (TRPV3), or Transient Receptor Potential Vaniloid 4 (TRPV4).
<7> The central nerve-activating composition according to any one of the above <1> to <6>, which is provided as a pharmaceutical product, a cosmetic product, or a food and beverage product.
<8> An agent for producing superoxide and/or hydrogen peroxide, comprising 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof.
<A> A method for activating the central nerve, comprising administering 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, to a subject who is in need of activation of the central nerve, and adjusting the autonomic nerve activity via production of superoxide andlor hydrogen peroxide.
<B> A method for producing superoxide and/or hydrogen peroxide, comprising administering 2-(3,4 -dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, to a subject.
<C> 2-(3,4-Dihydroxyphenyl)chromenylium-3,5,7-triol 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, which is for use in a treatment of activating the central nerve by adjusting the autonomic nerve activity via production of superoxide and/or hydrogen peroxide.
<D> 2(3,4-Dihydroxyphenylichromenylium-3,5,7-triol, 2-(3,4,5-trihydroxylphenykhromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, which is for use in a treatment of producing superoxide and/or hydrogen peroxide.
<E> Use of 2(3,4-dihydroxyphenyl)chromenylium-3,5,7-triol, 2-(3,4,5-trihydroxylphenyl)chrotnenylium-3,5,7-triol, or a compound in which 2 to 15 molecules of (2R,3R)-2(3,4-dihydroxyphenyl)-3,4-dihydro-2H-chromene-3,5,7-triol are condensed by a 4β→8 bond, or a salt, hydrate, solvate or polyrnorph thereof, for production of a central nerve-activating composition that adjusts the autonomic nerve activity via production of superoxide and/or hydrogen peroxide.
<F> Use of 2(3,4-dihydroxyphenyl)chromenylium-3,5,7-triol, 2-(3,4,5-trihydroxylphenylichromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, for production of an agent for producing superoxide and/or hydrogen peroxide.

### Advantageous Effects of Invention

The central nerve-activating composition of the present invention exhibits activating actions on the central nervous system, such as wakening, improvement of concentration, improvement of stress resilience and improvement of working memory, and adjusts the autonomic nerve function via the central nerve-activating actions, and exhibits actions such as neovascularization, wound healing, beiging of adipose cells, and skeletal muscle hypertrophic action, The central nerve-activating composition of the present invention is useful for improvement of cognitive function, wound healing, neovascularization, and the like. The central nerve-activating composition of the present invention is useful for prevention or treatment of depressive symptoms, sleep rhythm disorder, orthostatic dysregulation, mild cognitive impairment, dementia, chronic wounds, obesity, hypertension, hyperlipidemia, diabetes, sarcopenia, disuse muscular atrophy, and the like.

### BRIEF DESCRIPTION OF DRAWINGS

FIG. 1 shows the results obtained by measuring astringency, in a case where the compound of the present invention and commonly used analogous compounds were ingested.

FIG. 2 shows the results obtained by measuring locomotor activity, in a case where the compound of the present invention was administered once.

FIG. 3 shows the results obtained by performing a location recognition test and an object recognition test and the uptake amount of deoxybromouridine in the hippocampal dentate gyrus, in a case where the compound of the present invention was administered repeatedly.

FIG. 4 shows the results obtained by measuring avoidance behavior from stressors, a case where the compound of the present invention was repeatedly administered to social defeat stress animal models.

FIG. 5 shows the results obtained by calculating the 50% effective doses of the compound of the present invention and commonly used analogous compounds regarding blood flow-improving action, in a case where the compound of the present invention and commonly used analogous compounds were administered once.

FIG. 6 shows the results obtained by measuring the blood flow of the cremasteric arteriole, in a case where the compound of the present invention and/or a TRP channel inhibitor were administered once.

FIG. 7 shows the results obtained by measuring locomotor activity, in a case where the compound of the present invention was administered once to TRP channel-knockout mice.

FIG. 8 shows the results obtained by measuring the amount of hydrogen peroxide produced, in a case where the pH of a solution of the compound of the present invention was fluctuated.

FIG. 9 shows the results obtained by measuring the amount of hydrogen peroxide produced in a solution of the compound of the present invention.

FIG. 10 shows the results obtained by measuring locomotor activity, in a case wh.ere the compound of the present invention andlor a hydrogen peroxide scavenger were administered once.

FIG. 11 shows the results obtained by measuring the blood flow of the cremasteric arteriole, in a case where the compound of the present invention and/or a hydrogen peroxide scavenger were administered once.

FIG. 12 shows the results obtained by measuring the blood pressure, in a case where the compound of the present invention and/or a hydrogen peroxide scavenger were administered repeatedly.

FIG. 13 shows the results obtained by measuring a histological change in the adipose tissues of the groin and the expression level of uncoupling protein 1, in a case where the compound of the present invention was administered repeatedly.

FIG. 14 shows the results obtained by measuring a histological change in the soleus muscle and the muscle cross-sectional area, in a case where the compound of the present invention was administered repeatedly.

FIG. 15 shows the results obtained by measuring a histological change in the soleus muscle and the muscle cross-sectional area, in a case where the compound of the present invention was repeatedly administered to disuse muscular atrophy models.

FIG. 16 shows the results obtained by detecting neovascularization, in a case where the compound of the present invention was administered repeatedly.

### EMBODIMENTS OF CARRYING OUT THE INVENTION

Hereafter, the embodiments of the present invention will be described.

The present inventors have conducted a sensory test on, in particular, the following compounds that are ingredients broadly utilized as food products, among 7000 types of polyphenol compounds:
(2R,3R)-2-(3,4-dihydroxyphenyl)-3,4-dihydro-2H-chromene-3,5,7-triol and a compound in which (2R,3R)-2-(3,4-dihydroxyphenyl)-3,4-dihydro-2H-chromene-3,5,7-triol is condensed by a 4β→8 bond;
2-(3,4-dihydroxyphenyl)chromenylium-3,5,7-triol;
2-(3,4,5-trihydroxylphenyl)chromenylium-3,5,7-triol;
2-(3,4-hydroxyphenyl-3,5,7-trihydroxy-4H-1-benzopyran-4-one (quercetin);
(2R,3R)-5,7-dihydroxy-2-(3,4,5-trihydroxyphenyl)-3,4-dihydro-2H-chromen-3-yl] 3,4,5-trihydroxybenzoate (EGCG);
(2S)-5-hydroxy-2-(3-hydroxy-4-methoxyphenyl)-7-[(2S,3R,4S,5S,(R)-3,4,5-trihydroxy-6-[[(2R,3R,4R,5R,6S)-3,4,5- trihydroxy-6-methyloxan-2-yl]oxymethyl]oxan-2-yl]oxy-2,3-dihydrochromen-4-one (hesperidin);
7-hydroxy-3-(4-hydroxyphenyl)chromen-4-one (dadzein); and
(1E,6E)-1,7-bis(4-hydroxy-3-methoxyphenyl)hepta-1,6-diene-3,5-dione (curcumin).

As a result, it was clarified that, when a human orally ingested 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R(3R)-2-(3,4-dihydroxyphenyl)-3,4-dihydro-2H-chromene-3,5,7-triol are condensed by a 4β→8 bond (hereinafter, all of the above-described compounds are collectively referred to as “the compound of the present invention” at times), the human felt strong astringency, compared with other polyphenol compounds (Example 1).

The compound of the present invention exhibits strong astringency. Thus, the present inventor has orally administered these compounds to laboratory animals, and has then evaluated the spontaneous activity of the animals after the oral administration. As a result, the present inventor has found that the compound of the present invention has an action to significantly increase the amount of spontaneous activity and to significantly prolong the wakening state, and has confirmed that the compound of the present invention is effective for the treatment of sleep rhythm disorder and orthostatic dysregulation (Example 2).

The compound of the present invention is found to have an awakening action. Thus, the present inventors have repeatedly administered these compounds to laboratory animals, and have then conducted a location recognition test and an object recognition test. As a result, the present inventors have also found that the compound of the present invention has an action to induce nerve regeneration in the hippocampus and to improve spatial memory/working memory, namely, to improve concentration/cognitive function, and have confirmed that the compound of the present invention is effective for mild cognitive impairment and dementia (Example 3).

After administration of the compound of the present invention, the action to improve concentration/cognitive function was observed. Thus, the present inventors have repeatedly administered these compounds to social defeat stress animal models, and have then evaluated avoidance behavior from stressors. As a result, the present inventors have found that the avoidance behavior from stressors is suppressed, namely, the compound of the present invention has an action to improve stress resilience, and have confirmed that the compound of the present invention is effective for the improvement of depressive symptoms (Example 4).

In general, it has been known that promotion of the sympathetic nerve activity increases the amount of peripheral blood flow. Hence, the present inventor has administered the compound of the present invention at graduated doses to laboratory animals, and has then observed the influence of the compound of the present invention on the peripheral blood flow. The 50% effective dose (ED50) was calculated regarding a peripheral blood flow-increasing action. As a result, the 50% effective dose of a compound in which (2R,3R)-2-(3,4-dihydroxyphenyl)-3,4-dihydro-2H-chromene-3,5,7-triol was condensed by a 4β→8 bond was in the range of 1 to 30 μg/kg of body weight. On the other hand, the 50% effective dose of 2-(3,4-dihydroxyphenyl)chromenylium-3,5,7-triol or 2-(3,4,5-trihydroxylphenyl)chromenylium-3,5,7-triol was in the range of 50 to 100 μg/kg of body weight. In the case of other compounds, administration of the compounds at a dose of 1 to 1000 μg/kg of body weight did not change the blood flow, and the 50% effective dose could not be calculated (Example 5).

The present inventor has co-administered the compound of the present invention and a TRP channel blocker to laboratory animals, and has then observed the influence of the co-administration on the peripheral blood flow. As a result of the co-administration of the TRP channel blocker, the blood flow-increasing action exhibited by the compound of the present invention was significantly suppressed (Example 6). From these results, it was found that the compound of the present invention activates the TRP channel.

In addition, the present inventor has orally administered the compound of the present invention to TRP channel-knockout mice, and has then evaluated the spontaneous activity of the mice. As a result, a significant increase in the amount of spontaneous activity and an action to significantly prolong the wakening state, which are observed in wild-type mice, disappeared (Example 7).

It has been conceived that the compound of the present ins,ention would be likely to be a ligand of the TRP channel. Thus, the present inventor has analyzed the quantitative structure-activity relationship of the compound of the present invention with TRP agonists that had been developed so far. A correlation was not found between the existing TRP agonists and the compound of the present invention.

Differing from other transmembrane receptors, the TRP channel is not only activated by a specific bond with a ligand, but is also activated by stimulation such as temperature, machine, pH, osmotic pressure or oxidative stress (Non-Patent Document 8).

It has been assumed that the compound of the present invention further produces hydrogen peroxide from superoxide in the neutral pH range by the following chemical reaction mechanism (Non-Patent Document 1).

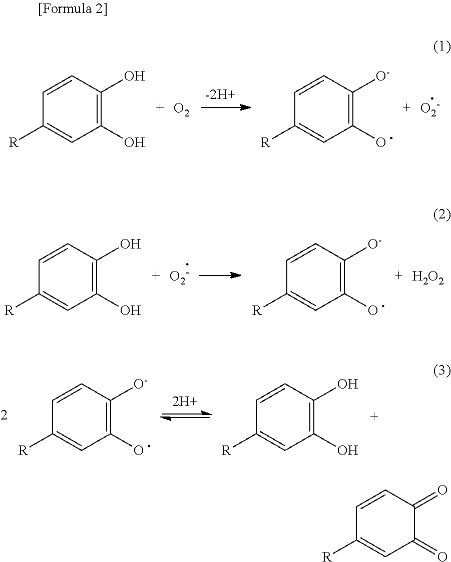

Hence, the present inventors have confirmed that the compound of the present invention significantly produces hydrogen peroxide in the neutral range (Examples 8 and 9).

Oral cavity, esophagus, small intestine and large intestine, from which stomach is excluded, or skin and mucous membrane, are weakly acidic (pH 6 to 7). Thus, it has been conceived that, when the compound of the present invention is exposed to alimentary canals excluding stomach, or to skin or mucous membrane, hydrogen peroxide would be further produced from superoxide, and would be recognized by the TRP channel expressed on the alimentary canal sensory nerve.

Hence, the present inventor has co-administered N-acetylcysteine that is a scavenger of superoxide and hydrogen peroxide, and the compound of the present invention, to laboratory animals, and has then evaluated the spontaneous activity of the animals. As a result, the present inventor has found that an increase in the amount of spontaneous activity observed upon single administration of the compound of the present invention is deleted, namely, that superoxide and/or hydrogen peroxide that are secondarily produced in the neutral range increase the amount of spontaneous activity (Example 10).

Moreover, the present inventor has co-administered N-acetylcysteine that is a scavenger of superoxide and hydrogen peroxide, and the compound of the present invention, to laboratory animals, and has then measured the amount of peripheral blood flow. As a result, the present inventor has found that an increase in the blood flow observed upon single administration of the compound of the present invention is deleted, namely, that superoxide and/or hydrogen peroxide that are secondarily produced in the neutral range increase the blood flow (Example 11).

It has been known that promotion of the sympathetic nerve activity increases the amount of peripheral blood flow, and that, as a result, it causes the improvement of vascular endothelial function and neovascularization and decreases the blood pressure. Hence, the present inventor has allowed laboratory animals to repeatedly ingest the compound of the present invention, and has then observed a change in the blood pressure after the repeated ingestion of the compound of the present invention. As a result, the present inventor has confirmed that the compound of the present invention exhibits a hypotensive action and is effective for hypertension (Example 12). On the other hand, when the compound of the present invention and N-acetylcysteine that is a scavenger of superoxide and hydrogen peroxide were co-administered to laboratory animals, such a hypotensive action was not observed (Example 12).

It has been known that promotion of the sympathetic nerve activity does not only increase the amount of peripheral blood flow, but the promotion of the sympathetic nerve activity also converts white adipose tissues to beige adipose tissues and increases heat production (Non-Patent Document 10). Thus, the present inventor has allowed laboratory animals to repeatedly ingest the compound of the present invention, has then excised the groin fat from the animals, and has then performed histological observation. As a result, the present inventor has found that the compound of the present invention converts white adipose tissues to beige adipose tissues, generates new blood vessels, and promotes heat production, and has confirmed that the compound of the present invention is effective for obesity, dyslipidemia, diabetes, and chronic wounds (Example 13).

It has been known that promotion of the sympathetic nerve activity promotes secretion of catecholamine from the adrenal gland into the blood, At the same time, it has been reported that adrenergic agonists promote differentiation and/or maturation of the skeletal muscle (Non-Patent Document 11). Hence, the present inventor has allowed laboratory animals to repeatedly ingest the compound of the present invention, has then excised the hind limb skeletal muscle from each animal, and has then measured the cross-sectional area of the skeletal muscle. As a result, the present inventor has found that the compound of the present invention has an action to promote muscular hypertrophy and a neovascularizati on action, and has confirmed that the compound of the present invention is effective for sarcopenia and chronic wounds (Example 14).

It has been known that disuse muscular atrophy occurs in an inactive state such as social withdrawal or frailty, and that mobility is lowered (Non-Patent Document 12). Hence, the present inventor has allowed disuse muscular atrophy animal models to repeatedly ingest the compound of the present invention, has then excised the hind limb skeletal muscle from each animal model, and has then measured the cross-sectional area of the skeletal muscle. As a result, the present inventor has found that the compound of the present invention suppresses disuse muscular atrophy (Example 15).

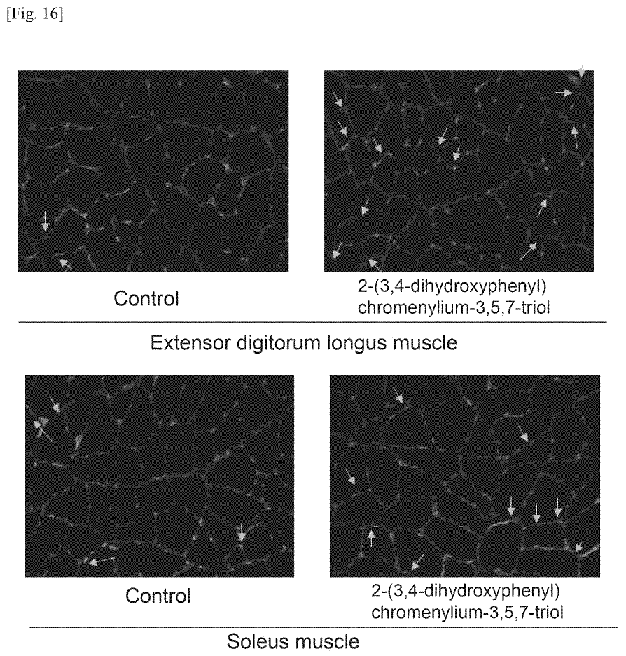

Furthermore, the present inventor has prepared a frozen section of the extensor digitorum longus muscle or the soleus muscle excised from each laboratory animal that had been repeatedly administered with the compound of the present invention, and has then detected CD31. serving as a marker of neovascularization according to an immunostaining method. As a result, significant expression of CD31 was observed, and a neovascularization image was confirmed (FIG. 16).
Non-Patent Document 10: Kajimura, S., Spiegelman, B. M. & Seale, P. Brown and Beige Fat: Physiological Roles beyond Heat Generation. Cell metabolism 22, 546-559.2015.
Non-Patent Document 11: Sato, S., Shirato, K., Tachivashiki, K. & Imaizumi, K. Muscle plasticity and beta(2)-adrenergic receptors: adaptive responses of beta(2)-adrenergic receptor expression to muscle hypertrophy and atrophy. J Biomed Biotechnol 2011, 729598, 2011.
Non-Patent Document 12: Bodine, S. C. Disuse-induced muscle wasting. The international journal of biochemistry & cell biology 45, 2200-2208, 2013.

The central nerve-activating composition of the present invention is a composition comprising, as an active ingredient, 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof (in the present description, these are collectively referred to as “the compound of the present invention” at times), wherein the composition is capable of adjusting the autonomic nerve activity via production of superoxide and/or hydrogen peroxide.

Examples of the compound in which 2 to 15 molecules of (2R,3R)-2-(3,4-dihydroxyphenyl)-3,4-dihydro-2H-chromene-3,5,7-triol are condensed by a 4β→8 bond may include the compounds as described above.

The salt of the above-described compound is not particularly limited, as long as it is a pharmaceutically acceptable salt. Examples of the salt may include inorganic acid salts, organic acid salts, metal salts, and ammonium salts. Citrulline is commercially available as a pharmaceutical product, a reagent or the like, and such commercially available products can be used. The salt is not particularly limited, as long as it is a pharmaceutically acceptable salt. Examples of the salt may include inorganic acid salts, organic acid salts, metal salts, and ammortium salts. Citrulline is commercially available as a pharmaceutical product, a reagent or the like, and such commercially available products can be used. Examples of the salt may include inorganic acid salts (e.g. salts with inorganic acids such as hydrochloric acid, hydrobromic acid, sulfuric acid, nitric acid, carbonic acid, or phosphoric acid), organic acid salts (e.g. salts with organic acids such as formic acid, acetic acid, maleic acid, fumaric acid, benzoic acid, ascorbic acid, lactic acid, succinic acid, bismethylenesalicylic acid, methanesulfonic acid, ethanedisulfonic acid, propionic acid, tartaric acid, malic acid, salicylic acid, citric acid, gluconic acid, aspartic acid, stead c acid, palmitic acid, itaconic acid, glycolic acid, p-aminobenzoic acid, glutamic acid, benzenesuifonic acid, cyclohexylsulfainic acid, methanesulfonic acid, ethanesulfonic acid, isethionic acid, or p-toluenesulfonic acid), metal salts (e.g. alkaline metal salts such as sodium and potassium; alkaline-earth metal salts such as magnesium, calcium, and barium; and metal salts including polyvalent metal salts such as aluminum), and ammonium salts (ammonium, tricyclohexylammonium, etc.).

Examples of the solvate of the above-described compound may include alcohol solvates such as ethanol solvate.

Examples of the polymorph of the above-described compound may include crystal polymorphs.

The central nerve-activating composition of the present invention produces hydrogen peroxide in one or more of the oral cavity, esophagus, small and large intestine, skin, and mucous membrane, then activates the sensory nerve as a result that the produced hydrogen peroxide is recognized by the superoxide and/or hydrogen peroxide domain of a transient receptor potential (TRP) expressed on the sensory nerve, and then promotes the central nerve activity via the secondary nerve, so that the central nerve-activating composition can promote the autonomic nerve activity.

The transient receptor potential (TRP) is preferably Transient Receptor Potential Ankyrin 1 (TRPA1), Transient Receptor Potential Melastatin 1 (TRPM1), Transient Receptor Potential Melastatin 2 (TRPM2), Transient Receptor Potential Cation channel 1 (TRPC1), Transient Receptor Potential Cation channel 3 (TRPC3), Transient Receptor Potential Cation channel4 (TRPC4), Transient Receptor Potential Cation channel 5 (TRPCS), Transient Receptor Potential Vaniloid 1 (TRPV1), Transient Receptor Potential Vaniloid 3 (TRPV3), or Transient Receptor Potential Vaniloid 4 (TRPV4).

The compound of the present invention is preferably administered by oral ingestion andlor application and/or aspiration. The compound of the present invention is administered to a human at a daily dose of 0.1 μg to 1000 mg, desirably 1 μg to 100 mg, and more desirably 10 μg to 10 mg by oral ingestion and/or application and/or aspiration, so that it can activate the central nerve.

The central nerve-activating composition of the present invention can be used for humans, for the purpose of achieving at least one of wakening, improvement of concentration, improvement of working memory, improvement of cognitive function, improvement of stress resilience, beiging of adipose tissues, muscular hypertrophy, and neovascularization, or for the purpose of preventing or treating at least one of sleep rhythm disorder, orthostatic dysregulation, depressive symptoms, mild cognitive impairment, dementia, disuse muscular atrophy, sarcopenia, obesity, hypertension, hyperlipidemia, and diabetes.

Further, the compound of the present invention can also be used as an agent for producing superoxide and/or hydrogen peroxide.

According to the present invention, provided is a method for activating the central nerve, comprising allowing a human or a non-human animal to ingest an effective amount of the compound of the present invention, or administering an effective amount of the compound of the present invention to a human or a non-human animal by oral administration and/or application andlor aspiration.

According to the present invention, provided is use of the compound of the present invention for production of a central nerve-activating composition. According to the present invention, provided is the compound of the present invention for use in a treatment for activation of the central nerve.

The central nerve-activating composition of the present invention can be provided as a pharmaceutical product, a quasi-drug, a cosmetic product, a food and beverage product, or a feed, and can be preferably provided as a pharmaceutical product or a food and beverage product or a cosmetic product.

The compound of the present invention can be administered to a human and a non-human animal by oral administration and/or application and/or aspiration. Examples of the oral agent may include granules, powder agents, tablets, pills, capsules, syrups, emulsions, suspensions, and liquid agents. The compound of the present invention can be formulated according to ordinary methods using pharmaceutically acceptable carriers. In addition, examples of the dosage form of the compound of the present invention for the application may include creams, pastes, ointments, gelling agents, emulsions, liquid agents, lotions, and spray agents. Also, the dosage -form of the compound of the present invention for the application may be a poultice, a taping agent or the like. Moreover, examples of the dosage form of the compound of the present invention for the aspiration may include aerosol formulations, dry powder formulations, and nebulizer formulations (i.e. liquid formulations, in which a drug is converted to a mist form, using a nebulizer, and is then sprayed for aspiration). These agents can also be formulated according to ordinary methods using pharmaceutically acceptable carriers. Examples of the pharmaceutically acceptable carriers may include excipients, binders, diluents, additives, flavors, buffering agents, thickeners, coloring agents, stabilizers, emulsifiers, dispersants, suspending agents, and antiseptics.

The administration method of the central nerve-activating composition of the present invention may be oral ingestion or oral administration, or may also be parenteral administration.

When the central nerve-activating composition of the present invention is provided as a food and beverage product, the compound of the present invention can be directly added into the food and beverage product. Examples of the foods may include health foods, functional foods, specific health use foods, nutrition function foods, foods with functional claims, infant foods, maternal foods, and sick foods. The compound of the present invention can also be provided by being contained in foods ingested on a daily basis or foods ingested as supplements.

The content of the compound of the present invention in the food and beverage product or the supplement is not particularly limited, and it may be, for example, 0.01% to 50% by mass, and preferably 0.1% to 5% by mass.

Examples of the food and beverage product may include: chocolate, cocoa; starch-based foods such as bread, biscuits, noodles, crackers, or nutrition bars; various types of confectioneries such as candies, gums, gummies, or snacks; milk and milk products, such as cow milk, processed milk, ice creams, fermented milk (yogurt, etc.), milk drinks, cheeses, butters, or creams; desserts such as pudding; jelly, bavarois, or mousse; beverages such as non-alcoholic beverages or alcoholic beverages; processed meat products such as hams or sausages; processed fish products such as kamaboko (a semi cylindrical sausage of boiled fish paste), chilaava (a tubular roll of boiled fish paste), or fish sausage; processed fruit products such as jam or puree; and condiments such as roux or sauce.

Furthermore, the central nerve-activating composition of the present invention can also be used as a cosmetic product. Examples of the cosmetic product may include emulsion, serum, lotion, microernulsion essence, pack, foundation, lipstick, eyeshadow, shampoo, conditioner, and bath additive. The central nerve-activating composition of the present invention can be used as cream, gel, emulsion, serum, lotion, pack, foundation, shampoo, conditioner, bath additive, microneedle, etc.

The central nerve-activating composition of the present invention can exhibit its effects by single ingestion. In some cases, however, the central nerve-activating composition of the present invention may be continuously administered or ingested for 1 week or more.

The present invention will be more specifically described in the following examples. However, these examples are not intended to limit the scope of the present invention.

### EXAMPLES

### (Example 1) Astringency

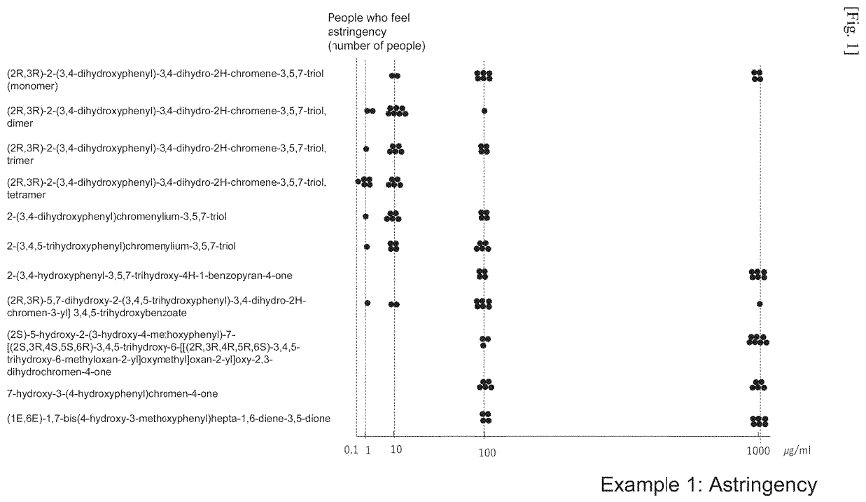

Ten healthy volunteers held in their oral cavity, solutions obtained by gradually diluting (2R,3R)-2-(3,4-dihydroxyphenyl)-3,4-dihydro-2H-chromene-3,5,7-triol, and a dimer, a trimer or a tetramer obtained by condensing the above compound by a 4β→8 bond, 2-(3,4-dihydroxyphenyl)chromenylium-3,5,7-triol or 2-(3,4,5-trihydroxylphenyl)chromenylium-3,5,7-triol, 2-(3,4-hydroxyphenyl-3,5,7-trihydroxy-4H-1-benzopyran-4-one (quercetin), (2R,3R)-5,7-dihydroxy-2-(3,4,5-trihydroxyphenyl)-3,4-dihydro-2H-chromen-3-yl] 3,4,5-trihydroxybenzoate (EGCG), (2S)-5-hydroxy-2-(3-hydroxy-4-methoxyphenyl)-7-[(2S,3R,4S,5S,6R)-3,4,5-trihydroxy-6-[[(2R,3R,4R,5R,6S)-3,4,5- trihydroxy-6-methyloxan-2-yl]oxymethyl]oxan-2-yl]oxy -2,3-dihydrochromen-4-one (hesperidin), 7-hydroxy-3-(4-hydroxyphenyl)chromen-4-one (daidzein), and (1E,6E)-1,7-bis(4-hydroxy-3-methoxyphenyl)hepta-1,6-diene-3,5-dione (curcumin), and the concentrations at which the volunteers felt astringency were then observed. As a result, it was found that the dimer, trimer, and tetramer of (2R,3R)-2-(3,4-dihydroxyphenyl)-3,4-dihydro-2H-chromene-3,5,7-triol and 2-(3,4-dihydroxyphenyl)chromenylium-3,5,7-triol or 2(3,4,5-trihydroxylphenyl)chromenylium-3,5,7-triol exhibit astringency at a concentration of 1 to 100 μg/ml, and in almost all cases, these compounds exhibit astringency at a concentration of 10 μg/ml, but that other polyphenol compounds exhibit astringency at a concentration of 100 to 1000 and in almost all cases, such other polyphenol compounds exhibit astringency at a concentration of 1000 μ/ml (FIG. 1).

### Example 2

To C57BL/6 male mice, purified water, or a dimer, a trimer or a tetramer obtained by condensing (2R,3R)-2-(3,4-dihydroxyphenyl)-3,4-dthydro-2H-chromene-3,5,7-triol by a 4β→8 bond, or 2-(3,4-dihydroxyphenyl)chromenylium-3,5,7-triol, was administered at a dose of 25 μg/kg of body weight, and thereafter, the mice were observed in an open field for 60 minutes.

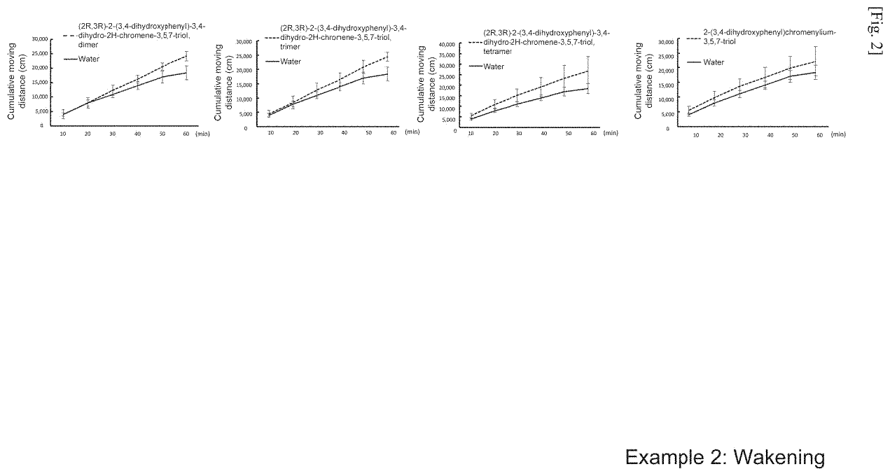

In the administration group with the dimer, trimer or tetramer obtained by condensing (2R,3R)-2-(3,4-dihydroxyphenyl)-3,4-dihydro-2H-chromene-3,5,7-triol by a 4β→8 bond, or with 2-(3,4-dihydroxyphenyl)chromenylium-3,5,7-triol, locomotor activity significantly increased, compared with a control group. Moreover, in the control group during this test, the animals repeated sleeping and wakening. In contrast, in the administration group with the dimer, trimer or tetramer obtained by condensing (2R,3R)-2-(3,4-dihydroxyphenyl)-3,4-dihydro-2H-chromene-3,5,7-triol by a 4β→8 bond, or with 2-(3,4-dihydroxyphenyl)chromenylium-3,5,7-triol, it was confirmed that the animals were in a wakening state (FIG. 2).

### Example 3 Steady State/Recognition Tests

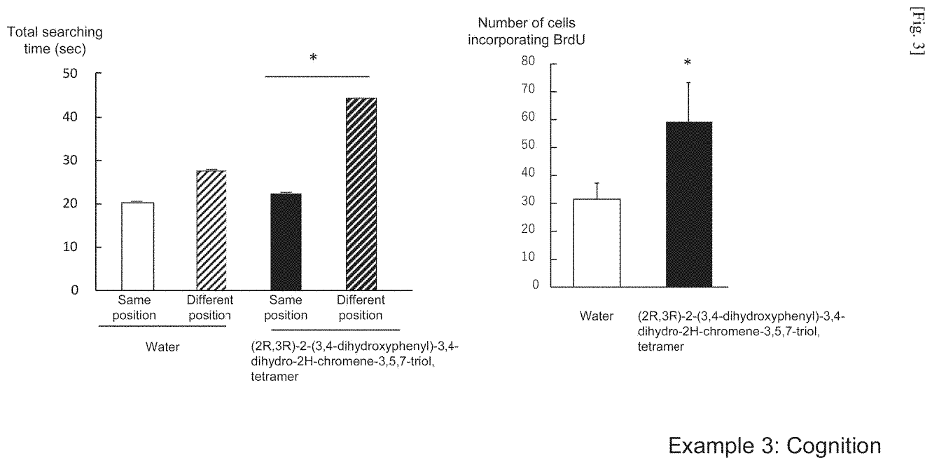

To C57BL/6 male mice, purified water, or atetramer obtained by condensing (2R,3R)-2-(3,4-dihydroxyphenyl)-3,4-dihydro-2H-chromene-3,5,7-triol by a 4β→8 bond at a dose of 25 μg/kg of body weight, and bromodeoxyuridine, were orally administered for 10 days. On the final administration day, the mice were forced to recognize two objects in a field for 10 minutes. Twenty minutes later, the mice were subjected to a location recognition test, and further 20 minutes after the location recognition test, the mice were subjected to an object recognition test. Thereafter, the mice were dissected, and whole brain was excised from each mouse, and a frozen section thereof was then prepared. The location recognition test was carried out as an indicator of spatial memory. As a result, it was found that the results of a group administered with the tetramer obtained by condensing (2R,3R-2-(3,4-dihydroxyphenyl)-3,4-dihydro-2H-chromene-3,5,7-triol by a 4β→8 bond were significantly improved, compared with the purified water administration group. Moreover, the uptake amount of deoxybromouridine as an indicator of neurogenesis in the hippocampus/dentate gurus significantly increased in the administration group, compared with the control group, and thus, the compound was found to have a neuron-generating action (FIG. 3).

### Example 4 Social Defeat Stress Models/Recognition Test

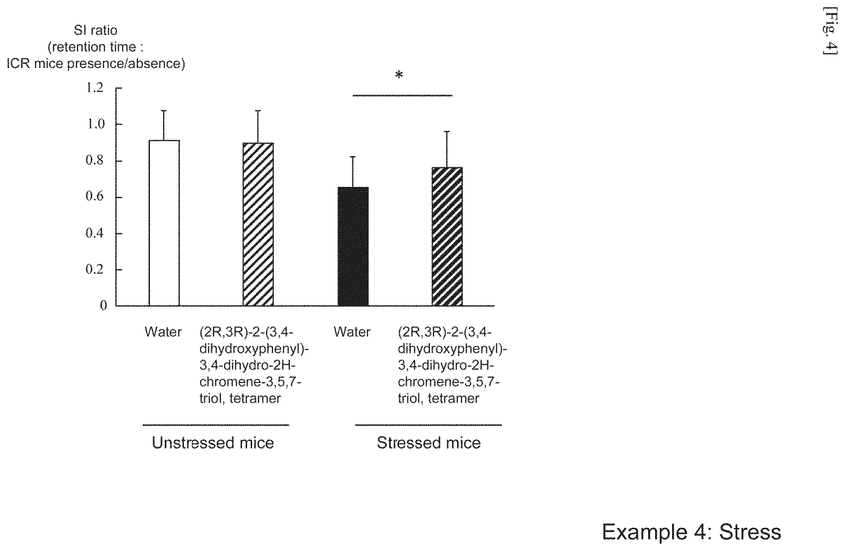

To C57BL/6 male mice, purified water, or atetramer obtained by condensing (2R,3R)-2-(3,4-dihydroxyphenyl)-3,4-dihydro-2H-chromene-3,5,7-triol by a 4β→8 bond at a dose of 25 μg/kg of body weight, was repeatedly administered by oral administration. At the same time, these mice were forced to invade a home cage for ICR male mice for 10 minutes, so that physical stress was imposed to the C57BL/6 male mice. Thereafter, the C57BL/6J mice were transferred to the opposite side of a transparent partition plate and were forced to live there for 24 hours, so that mental stress was imposed to the mice. These operations were repeated for 10 days, so as to produce social defeat stress models. On the final administration day, a transparent partition was established in an open field test, and the behavior of the C57BL/6 male mice was evaluated in a case where the ICR mice were present or were not present. The retention time around the transparent partition in the case where the ICR mice were not present was defined as a denominator, and the retention time around the transparent partition in the case where the ICR mice were present was defined as a numerator, so as to obtain a ratio (SI ratio). As a result, when compared with an unstressed mice/purified water administration group, the SI ratio was significantly reduced in the stressed mice/purified water administration group. However, the tetramer obtained by condensing (2R,3R-2-(3,4-dihydroxyphenyl)-3,4-dihydro-2H-chromene-3,5,7-triol by a 4β→8 bond suppressed this reduction. That is to say, it was observed that the tetramer obtained by condensing (2R,3R)-2-(3,4-dihydroxyphenyl)-3,4-dihydro-2H-chromene-3,5,7-triol by a 4β→8 bond improved stress resilience (FIG. 4).

### Example 5 Blood Flow LD50

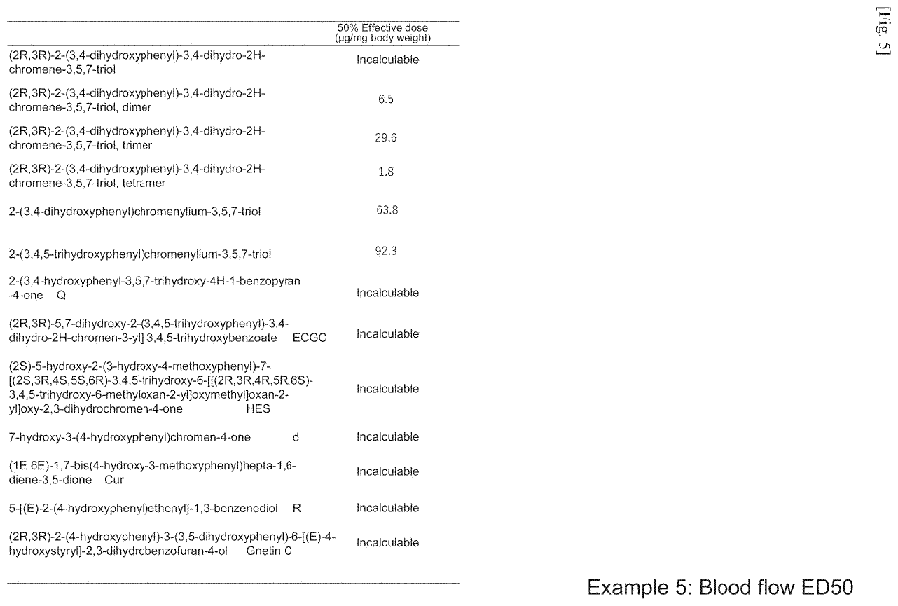

Cannula was inserted into the stomach of Wistar male rats under anesthesia, and the cremaster muscle was prepared, Not only a dimer, a trimer or a tetramer obtained by condensing (2R,3R)-2-(3,4-dihydroxyphenyl)-3,4-dihydro-2H-chromene-3,5,7-triol by a 4β→8 bond, 2-(3,4-dihydroxylphenyl)chromenylium-3,5,7-triol, or 2-(3,4,5-trihydroxyphenyl)chromenylium-3,5,7-triol, but also (2R,3R)-2-(3,4-dihydroxyphenyl)-3,4-dihydro-2H-chromene-3,5,7-triol, 2-(3,4-hydroxyphenyl-3,5,7-trihydroxy-4H-1-benzopyran-4-one (quercetin), (2R,3R)-5,7-dihydroxy-2-(3,4,5-trihydroxyphenyl)-3,4-dihydro-2H-chromen-3-yl] 3,4,5-trihydroxybenzoate (EGCG), (2S)-5-hydroxy-2-(3-hydroxy-4-methoxyphenyl)-7-[(2S,3R,4S,5S,6R)-3,4,5-trihydroxy-6-[[(2R,3R,4R,5R,6S)-3,4,5-trihydroxy-6-methylaxan-2-yl]oxymethyl]oxan-2-yl]oxy-2,3-dihydrochromen-4-one (hesperidin), 7-hydroxy-3(4-hydroxyphenyl)chromen-4-one (daidzein), (1E,6E)-1,7-bis(4-hydroxy-3-methoxyphenyl)hepta-1,6-diene-3,5-dione (curcumin), 5-[(E)-2-(4-hydroxyphenypenyl)ethenyl]-1,3-benzenediol (resveratrol), and (2R,3R)-2-(4-hydroxyphenyl)-3-(3,5-dihydroxyphenyl)-6-[(E)-4-hydroxystyryl]-2,3-dihydrobenzofuran-4-ol (Gnefin C), were administered to the rats at doses of 1, 10, 100, and 1000 μg/kg of body weight. A fluctuation in the blood flow of the cremasteric arteriole 60 minutes after the administration was measured using a laser Doppler blood-flowmeter. The dimer, trimer or tetramer obtained by condensing (2R,3R)-2(3,4-dihydroxyphenyl)-3,4-dihydro-2H-chromene-3,5,7-triol by a 4β→8 bond, 2-(3,4 -dihydroxyphenyl)chromenylium-3,5,7-triol, or 2-(3,4,5-trihydroxylphenyl)chromenylium-3,5,7-triol exhibited a dose-dependent blood flow-increasing action. However, no changes were observed in the case of other compounds. From these data, the 50% effective dose of each compound was calculated. The 50% effective dose of the compound obtained by condensing (2R,3R)-2-(3,4-dihydroxyphenyl)-3,4-dihydro-2H-chromene-3,5,7-triol by a 4β→8 bond was in the range of 1 to 30 μg/kg of body weight, whereas the 50% effective dose of 2-(3,4-dihydroxyphenyl)chromenylium-3,5,7-triol or 2-(3,4,5-trihydroxylphenyl)chromenylium-3,5,7-triol was in the range of 50 to 100 μg/kg of body weight. In other compounds, no changes were observed in the blood flow when such other compounds were administered at a dose of 1, 10, 100, or 1000 μg/kg, and thus, the 50% effective dose could not be calculated (FIG. 5).

### Example 6 Blood Flow Blocker

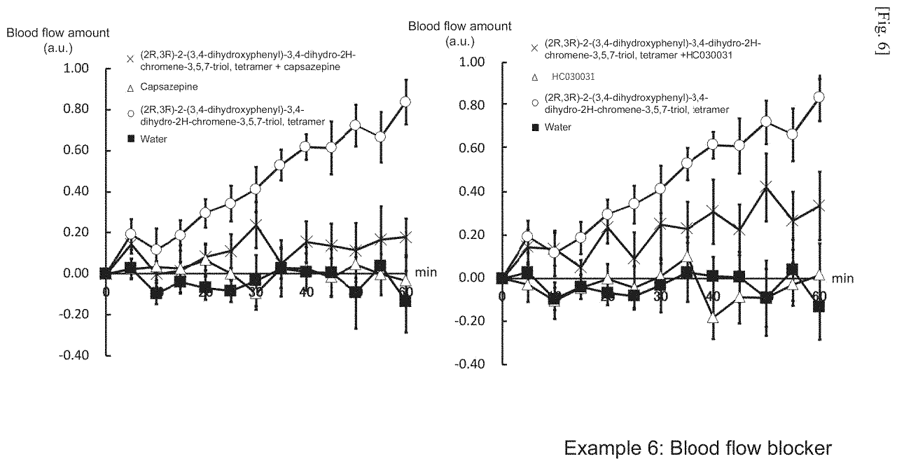

Cannula was inserted into the stomach of Wistar male rats under anesthesia, and the cremaster muscle was prepared. Capsazepine (3 mg/kg of body weight) was used as a TRPV1 blocker, and HC-030031 (0.1 mg/kg of body weight) was used as a TRPA1 blocker. A tetramer obtained by condensing (2R,3R)-2-(3,4-dihydroxyphenyl)-3,4-dihydro-2H-chromene-3,5,7-triol by a 4β→8 bond (10 μg/kg of body weight), and capsazepine (3 mg/kg of body weight) used as a TRPV1 blocker or HC-030031 (0.1 mg/kg of body weight) used as a TRPA1 blocker, were co-administered to laboratory animals, and the influence on the peripheral blood flow was then observed. By co-administration with capsazepine or HC-030031, the blood flow-increasing action exhibited by the tetramer obtained by condensing (2R,3R)-2-(3,4-dihydroxyphenyl)-3,4-dihydro-2H-chromene-3,5,7-triol by a 4β→8 bond was significantly suppressed (FIG. 6).

### Example 7 Wakening KO Mice

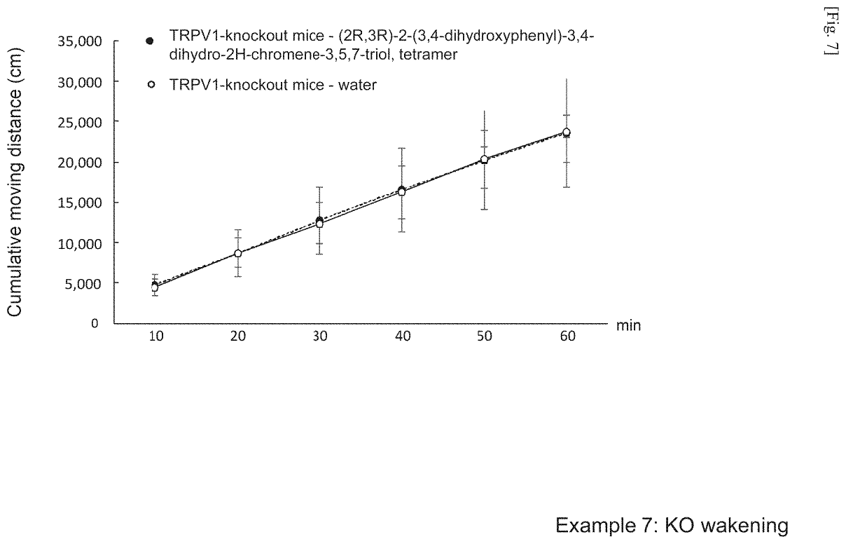

A tetramer obtained by condensing (2R,3R-2-(3,4-dihydroxyphenyl)-3,4-dihydro-2H-chromene-3,5,7-triol by a 4β→8 bond was orally administered at a dose of 25 μg/kg of body weight to TRPV1-knockout mice, and the mice were then observed in an open field for 60 minutes. An increase in the locomotor activity and extension of the wakening state, which had been observed in wild-type mice, were not observed (FIG. 7).

### Example 8 In vitro H₂O₂ Production (1)

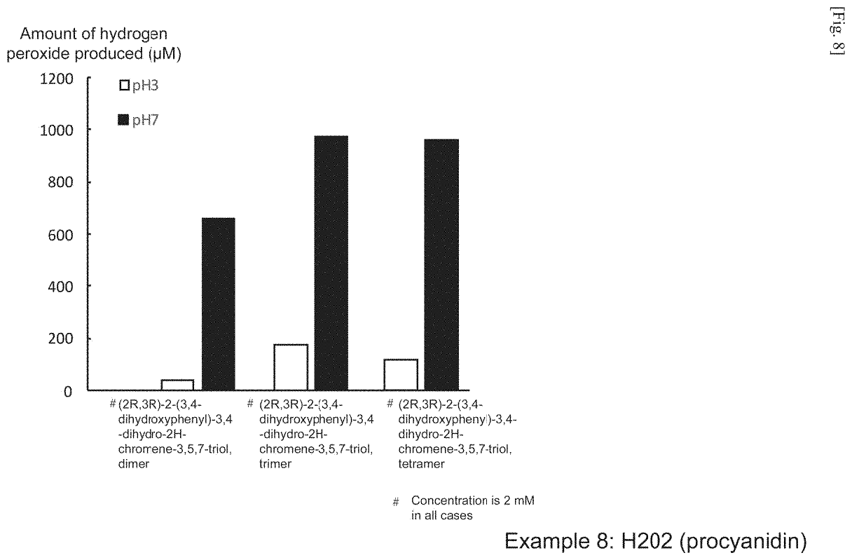

The amount of hydrogen peroxide produced by a dimer, a trimer or a tetramer obtained by condensing (2R,3R)-2-(3,4-dihydroxyphenyl)-3,4-dihydro-2H-chromene-3,5,7-triol by a 4β→8 bond at pH 3.0 and at pH 7.0 was measured by applying an FOX assay method, in which the coloration reaction of Fe³⁺-xylenol orange was utilized. The dimer, trimer or tetramer obtained by condensing (2R,3R)-2-(3,4-dihydroxyphenyl)-3,4-dihydro-2H-chromene-3,5,7-triol by a 4β→8 bond produced hydrogen peroxide. The produced amount of hydrogen peroxide was significantly increased at pH 7, compared with at pH 3 (FIG. 8).

### Example 9 In vitro H₂O₂ (Anthocyanin)

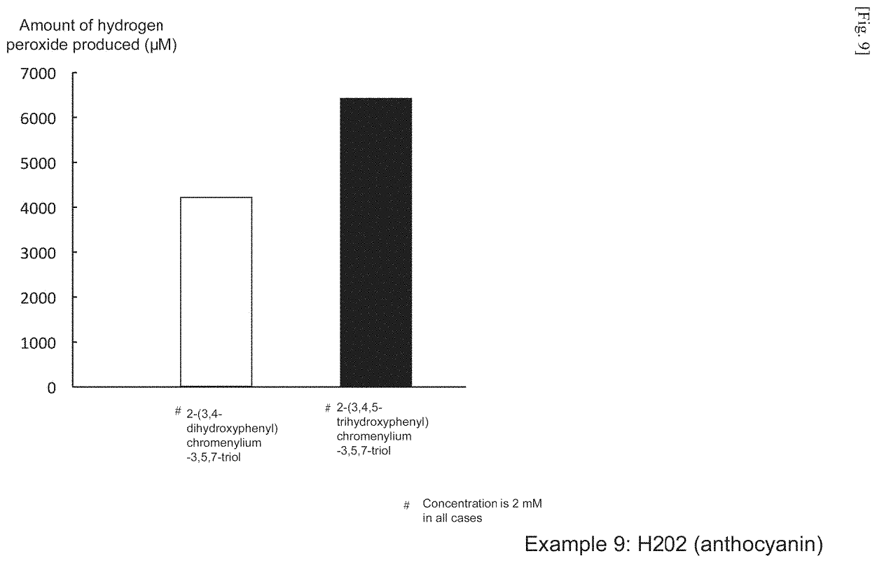

The amount of hydrogen peroxide produced by 2-(3,4-dihydroxyphenyl)chromenylium-3,5,7-triol or 2-(3,4,5-trihydroxylphenyl)chromenylium-3,5,7-triol at pH 7.0 was measured by applying an FOX assay method, in which the coloration reaction of Fe³⁺-xylenol orange was utilized. As a result, 2-(3,4-dihydroxyphenyl)chromenylium-3,5,7-triol and 2-(3,4,5-trihydroxylphenyl)chromenylium-3,5,7-triol significantly produced hydrogen peroxide (FIG. 9).

### Example 10 Wakening NAC

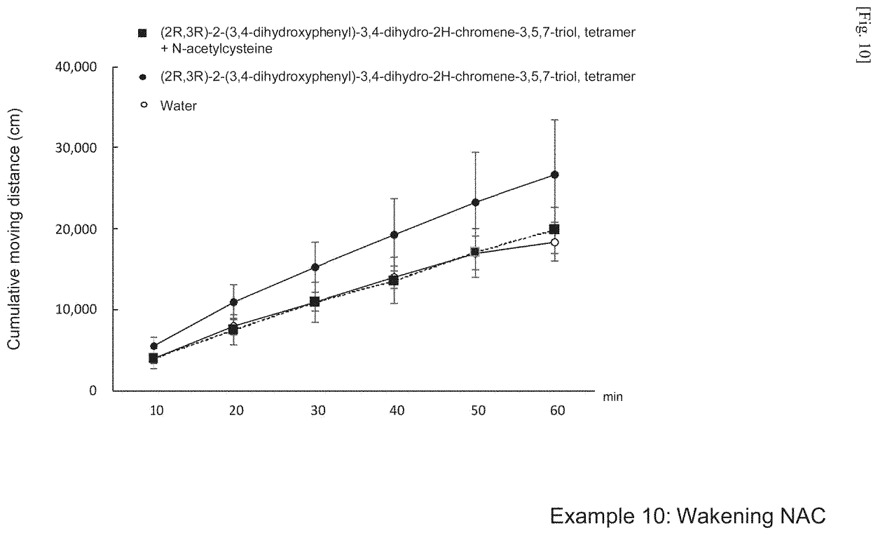

A tetramer of (2R,3R)-2-(3,4-dihydroxyphenyl)-3,4-dihydro-2H-chromene-3,5,7-triol (25 μg/kg of body weight) and N-acetylcysteine (100 mg/kg of body weight) were co-administered to laboratory animals, and the animals were then observed in an open field for 60 minutes. As a result, the locomotor activity-increasing action of the tetramer of (2R,3R)-2-(3,4-dihydroxyphenyl)-3,4-dihydro-2H-chromene-3,5,7-triol was not observed due to co-administration with N-acetylcysteine (FIG. 10).

### Example 11 Blood Flow NAC

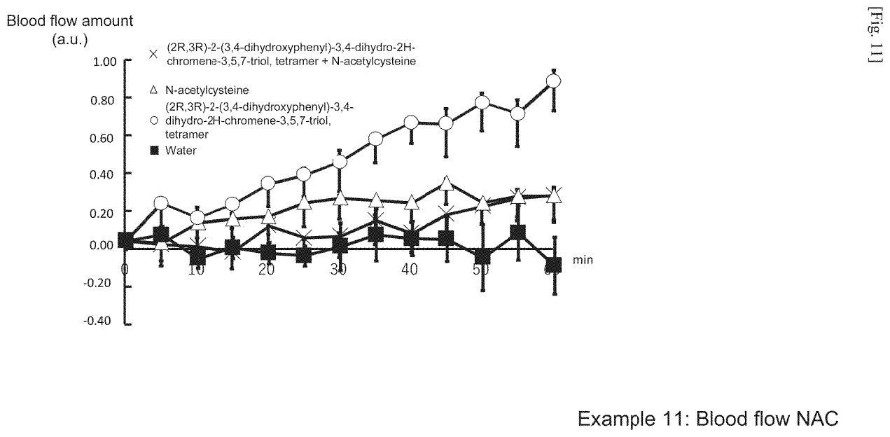

Cannula was inserted into the stomach of Wistar male rats under anesthesia, and the cremaster muscle was prepared. A tetramer obtained by condensing (2R,3R)-2-(3,4-dihydroxyphenyl)-3,4-dihydro-2H-chromene-3,5,7-triol by a 4β→8 bond (10 μg/kg of body weight) and N-acetylcysteine (100 mg/kg of body weight) were co-administered to laboratory animals, and a fluctuation in the blood flow of the cremasteric arteriole 60 minutes after the administration was measured using a laser Doppler blood-flowmeter. By single administration of the tetramer obtained by condensing (2R,3R)-2-(3,4-dihydroxyphenyl)-3,4-dihydro-2H-chromene-3,5,7-triol by a 4β→8 bond (10 μg/kg of body weight), as significant increase in the blood flow amount of the cremasteric arteriole was observed. However, this increase was disappeared by co-administration with N-acetylcysteine (FIG. 11).

### Example 12 Blood Pressure NAC

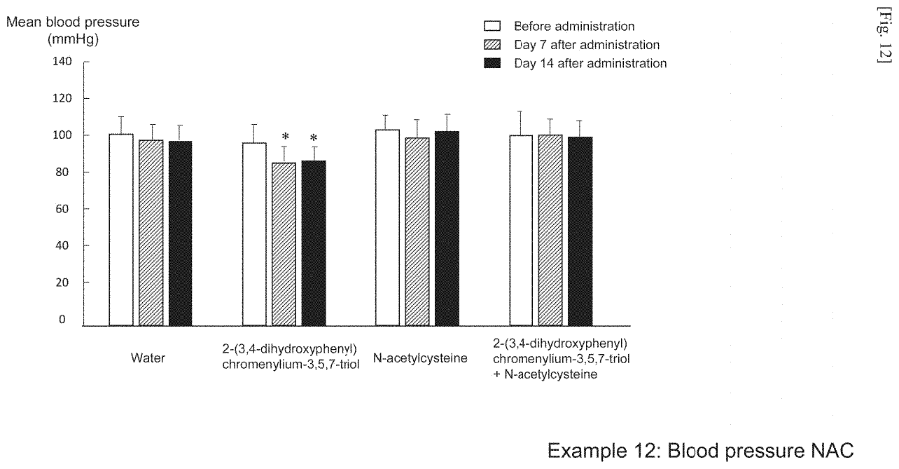

Wistar male rats were divided into 4 groups, and thereafter, 2-(3,4-dihydroxyphenyl)-3-(D-glucopyranooxyl)chromenylium-3,5,7-triol (1 mg/kg of body weight) and/or N-acetylcysteine (100 mg/kg of body weight) were co-administered to the laboratory animals for 14 days. Thereafter, the blood pressure of the animals was measured over time, using a non-heated type sphygmomanometer. 2-(3,4-Dihydroxyphenyl)-3-(D-glucopyranooxypchromenylium-3,5,7-triol significantly decreased the blood pressure on Day 7 and Day 14 after the administration. However, this change was disappeared by co-administration with N-acetylcysteine (FIG. 12).

### Example 13 Adipose Cells

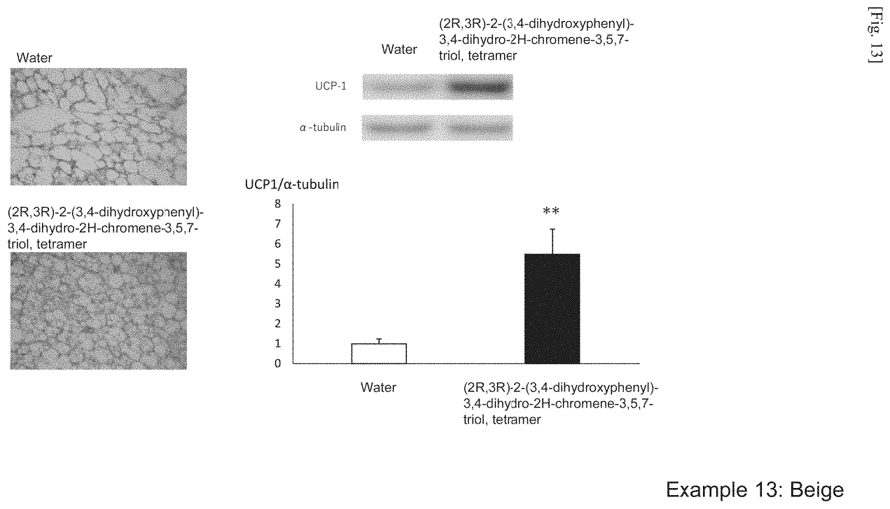

C57BL/6 male mice were divided into 2 groups, and thereafter, purified water or a tetramer obtained by condensing (2R,3R)-2-(3,4-dihydroxyphenyl)-3,4-dihydro-2H-chromene-3,5,7-triol by a 4β→8 bond (25 μg/kg of body weight) was repeatedly administered to the mice by oral administration. Two weeks later, the nice were dissected, the groin fat was then excised from each mouse, and a frozen section thereof was then prepared. The section was stained with HE, and was then subjected to histological observation. As a result, in a control group, white adipose tissue image, in which almost all cells were filled with lipid droplets, was shown. On the other hand, in the administration group with the tetramer obtained by condensing (2R,3R)-2-(3,4-dihydroxyphenyl)-3,4-dihydro-2H-chromene-3,5,7-triol by a 4β→8 bond, a reduction in the cell area due to the decrease of lipid droplets and the browning of the cells, and also a neovascularization image were observed. In addition, the expression level of uncoupling protein 1 serving as a differentiation marker into beige fats was significantly increased (FIG. 13).

### Example 14 Muscular Hypertrophy

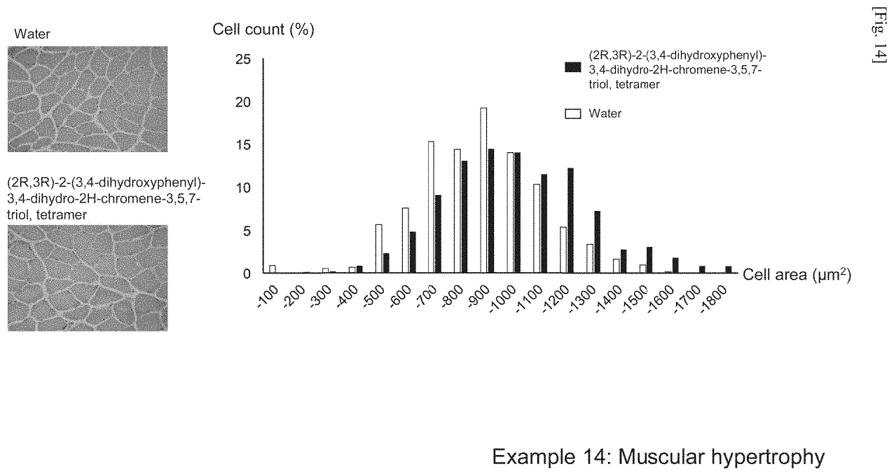

C57BL/6 male mice were divided into 2 groups, and thereafter, purified water or a tetramer of (2R,3R)-2-(3,4-dihydroxyphenyl)-3,4-dihydro-2H-chromene-3,5,7-triol (25 μg/kg of body weight) was repeatedly administered to the mice by oral administration. Two weeks later, the mice were dissected, the soleus muscle was then excised from each mouse, and a frozen section thereof was then prepared. The section was stained with HE, and was then subjected to histological observation, and at the same time, the muscle cross-sectional area was measured. As a result, in the administration group with the tetramer of (2R,3R)-2-(3,4-dihydroxyphenyl)-3,4-dihydro-2H-chromene-3,5,7-triol , the muscle cross-sectional area was significantly increased and a neovascularization image was seen, compared with a control group (FIG. 14).

### Example 15 Disuse Muscular Atrophy

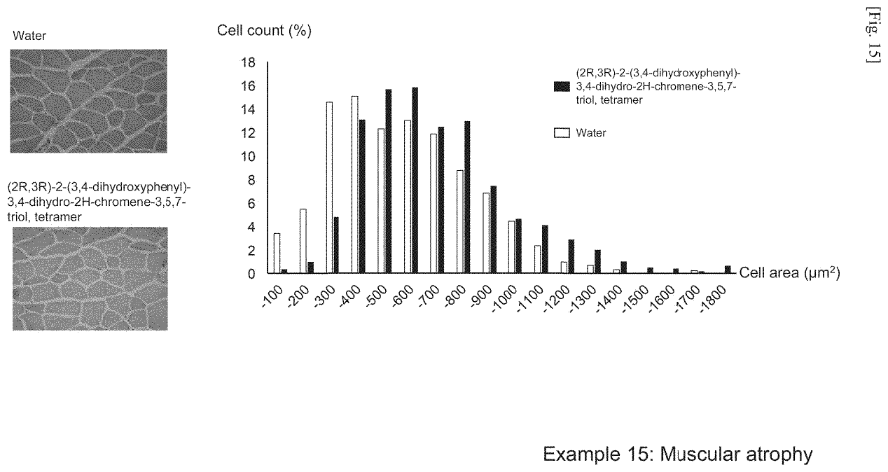

C57BL/6 male mice were divided into 2 groups, and were then subjected to a tail suspension treatment. Thereafter, purified water or a tetramer of (2R,3R)-2-(3,4-dihydroxyphenyl)-3,4-dihydro-2H-chromene-3,5,7-triol (25 μg/kg of body weight) was repeatedly administered to the mice by oral administration for 2 weeks. Two weeks later, the mice were dissected, the soleus muscle was then excised from each mouse, and a frozen section thereof was then prepared. The section was stained with HE, and was then subjected to histological observation, and at the same time, the muscle cross-sectional area was measured. As a result, in a control group, a significant reduction in the muscle cross-sectional area was observed, but in the administration group with the tetramer of (2R,3R)-2-(3,4-dihydroxyphenyl)-3,4-dihydro-2H-chromene-3,5,7-triol, such a change was significantly suppressed (FIG. 15).

### Example 16 Neovascularization

Wistar male rats were divided into 2 groups, and thereafter, a tetramer of 2-(3,4-dihydroxyphenyl)-3-(D-glucopyranooxyl)chromenylium-3,5,7-triol (1 mg/kg of body weight) was co-administered. to the laboratory animals for 14 days, Thereafter, the animals were subjected to dissection. A frozen section was prepared from the excised extensor digitorum longus muscle or soleus muscle, and CD31 serving as a neovascularization marker was then detected by applying an immunostaining method. As a result, in the administration group with 2-(3,4-dihydroxyphenyl)-3-(D-glucopyranooxyl)chromenylium-3,5,7-triol, a significant expression of CD31 was observed, and a neovascularization was confirmed, compared with a control group (FIG. 16).

1-8. (canceled)

9. A method for activating the central nerve, comprising administering 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, to a subject who is in need of activation of the central nerve, and adjusting the autonomic nerve activity via production of superoxide and/or hydrogen peroxide.

10. The method according to claim 9, which is used for the purpose of achieving at least one of wakening, improvement of concentration, improvement of working memory, improvement of cognitive function, improvement of stress resilience, beiging of adipose tissues, muscular hypertrophy, and neovascularization, or for the purpose of preventing or treating at least one of sleep rhythm disorder, orthostatic dysregulation, depressive symptoms, mild cognitive impairment, dementia, disuse muscular atrophy, sarcopenia, obesity, hypertension, hyperlipidemia, and diabetes.

11. The method according to claim 9, wherein 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof is administered by oral ingestion and/or application and/or aspiration.

12. The method according to claim 9, wherein 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof is administered to a human at a daily dose of 0.1 μg to 1000 mg.

13. The method according to claim 9, which produces hydrogen peroxide in one or more of the oral cavity, esophagus, small and large intestine, skin, and mucous membrane, then activates the sensory nerve as a result that the produced hydrogen peroxide is recognized by the superoxide/hydrogen peroxide domain of a transient receptor potential (TRP) expressed on the sensory nerve, and then promotes the central nerve activity via the secondary nerve, so that the central nerve-activating composition promotes the autonomic nerve activity.

14. The method according to claim 13, wherein the transient receptor potential (TRP) is Transient Receptor Potential Ankyrin 1 (TRPA1), Transient Receptor Potential Melastatin 1 (TRPM1), Transient Receptor Potential melastatin 2 (TRPM2), Transient Receptor Potential Cation channel 1 (TRPC1), Transient Receptor Potential Cation channel 3 (TRPC3), Transient Receptor Potential Cation channel4 (TRPC4), Transient Receptor Potential Cation channel 5 (TRPCS), Transient Receptor Potential Vaniloid 1 (TRPV1), Transient Receptor Potential Vaniloid 3 (TRPV3), or Transient Receptor Potential Vaniloid 4 (TRPV4).

15. The method according to claim 9, wherein 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof is provided as a pharmaceutical product, a cosmetic product, or a food and beverage product.

16. A method for producing superoxide and/or hydrogen peroxide, comprising administering 2-(3,4-dihydroxyphenyl)chromenylium-3,5,7-triol, 2-(3,4,5-trihydroxylphenyl)chromenylium-3,5,7-triol, or a compound in which 2 to 15 molecules of (2R,3R)-2-(3,4-dihydroxyphenyl)-3,4-dihydro-2H-chromene-3,5,7-triol are condensed by a 4β→8 bond, or a salt, hydrate, solvate or polymorph thereof, to a subject.
